(19) United States
(12) Patent Application Publication
(10) Pub. No.: US 2014/0343339 A1
(43) Pub. Date: Nov. 20, 2014

(54) METHOD FOR OBTAINING OLEFINS FROM FURNACE GASES OF STEEL WORKS

(71) Applicants: Nicole Schodel, Munchen (DE); Ernst Haidegger, Riemerling (DE); Holger Schmigalle, Wolfratshausen (DE); Volker Goke, Geretsried (DE); Harald Schmaderer, Wolfratshausen (DE)

(72) Inventors: Nicole Schodel, Munchen (DE); Ernst Haidegger, Riemerling (DE); Holger Schmigalle, Wolfratshausen (DE); Volker Goke, Geretsried (DE); Harald Schmaderer, Wolfratshausen (DE)

(73) Assignee: LINDE AKTIENGESELLSCHAFT, Muenchen (DE)

(21) Appl. No.: 14/345,417

(22) PCT Filed: Aug. 7, 2012
(86) PCT No.: PCT/EP2012/003371

§ 371 Date: May 20, 2014

(30) Foreign Application Priority Data
Sep. 15, 2011 (DE) 10 2011 113 547.6
Nov. 17, 2011 (DE) 11009118.8

(51) Int. Cl. C07C 41/01; C07C 1/207

(52) U.S. Cl. CPC C07C 41/01; C07C 1/2076
USPC 585/639

(57) ABSTRACT

The invention relates to a method for processing furnace gas (4) from a steel and/or iron works, wherein said furnace gas (4) contains carbon dioxide and/or carbon monoxide and is at least partially integrated into a method (7) for the formation of dimethyl ether in conjunction with a hydrogen-containing gas (2), whereby a DME-containing product gas (8) is formed. At the outset of the method (7) for forming dimethyl ether, a ratio of hydrogen to carbon monoxide, weighted with the carbon dioxide concentration (formula (I)), of 0.9 to 1.1 is set and dimethyl ether is formed. The DME-containing product gas (8) is integrated into a method (9) for converting dimethyl ether to olefins, whereby an olefin-containing product gas (10) is formed, and wherein olefins (12), in particular ethylene and/or propylene, is/are separated from the olefin-containing product gas (10) by means of separating methods (11).

The invention relates to a process for processing offgas from a steelworks and/or a smelting works, where the offgas contains carbon monoxide and/or carbon dioxide.

In a smelting works, iron is obtained from iron ore by reduction using a blast furnace. As reducing agent, use is made essentially of coke. The blast furnace has a shaft construction and is supplied from the top alternately with a Möller layer (a mixture of iron ore and additives) and a layer of coke. Temperatures in the range from 2000° C. to 200° C. prevail in the blast furnace, with the temperature decreasing from the bottom upward.

The coke, which consists essentially of carbon, reacts strongly exothermically with oxygen to give carbon dioxide and thus generates temperatures in the range from 1800° C. to 2000° C. at the bottom of the blast furnace, when using pure oxygen up to 2200° C. The exothermic reaction is followed directly by the two endothermic reactions to form carbon monoxide:
CO₂+C→2 CO
H₂O+C→CO+H₂

Carbon monoxide and hydrogen serve as reducing agents in the blast furnace and reduce the iron oxides of the iron ore to iron and also reduce the oxides of the elements manganese, silicon and phosphorus which accompany iron. However, the iron takes up a proportion of carbon during the process.

The iron loaded with carbon is obtained as pig iron from the bottom of the blast furnace. At the upper end of the blast furnace (top), an offgas which is also referred to as top gas or blast furnace gas correspondingly collects. This offgas consists essentially of carbon oxides (carbon monoxide, carbon dioxide, each in a proportion of 15-25% by volume, nitrogen (50-60% by volume) and also 3% by volume of hydrogen, 0.5-1% by volume of methane and water), together with further trace elements. Such an offgas is to be treated by the process of the invention.

The pig iron formed in the blast furnace cannot be forged because of the carbon content and is processed further in a steelworks. The main process there is burning the carbon out of the iron in a converter. This process is also referred to as freshing. Usually, pure oxygen is blown onto and/or into the hot pig iron via one or more suitable nozzle lances in the converter. Here, the carbon is oxidized to carbon dioxide and liquid steel is formed as a result of the high temperatures. An offgas which consists mainly of carbon dioxide (about 20% by volume) and carbon monoxide (about 55% by volume) together with hydrogen (about 4% by volume) and whose treatment is likewise subject matter of the present invention is thus formed in the converter, too.

Smelting works and steelworks are frequently combined in one plant and referred to as steelworks. In this case, both the offgas from the blast furnace and the offgas from the converter or both together can be treated according to the present invention.

In most cases, a coking plant in which coke is produced from coal in a coke oven is also integrated into such plants. The coke required in the blast furnace is produced in coking plants which are frequently integrated into the smelting works or steelworks. In a coke oven, the volatile constituents in the coal are pyrolyzed by heating to a temperature of from 900° C. to 1400° C., liberated and extracted. This forms the coke consisting essentially of carbon and an offgas referred to as coking plant gas which contains the volatile constituents. The pyrolysis in the coke oven takes place in the absence of oxygen. The coking plant gas formed contains hydrogen (about 55%), methane, nitrogen, carbon monoxide, carbon dioxide, sulfur and higher hydrocarbons.

The two abovementioned types of plant are mentioned here merely by way of example. The invention is therefore not restricted to these types of plant. The invention is directed generally to the treatment of offgases from smelting works and/or steelworks which comprise carbon dioxide and/or carbon monoxide. That is to say, the offgases from steelworks or smelting works in which the reduction of the iron ore is effected by means of electric arc furnaces, direct reduction or similar processes are also encompassed by the present invention, provided that they contain carbon monoxide and/or carbon dioxide. The invention serves generally to treat offgases from metallurgical furnaces such as a converter.

A process for treating such an offgas is described, for example, in the US document US2011/0041517. According to the prior art disclosed here, the hot offgas from a metallurgical furnace is reformed by addition of a reducing agent, with the reducing agent being added when the oxygen concentration is 1% by volume or less and the reforming being considered to be complete when the temperature of the offgas is 800° C. or above. Here, the hot offgas is said to be cooled, the emission of carbon dioxide into the environment is said to be minimized and the offgas is said to attain a higher joule value, since such offgases are, according to the prior art, fed to a gas turbine for generating electric energy.

WO 2009/023987, CN 101 913 558, CN 101 823 937 and CN 102 079 689 disclose processes for preparing methanol or dimethyl ether from coking plant offgas and from offgas obtained in steel production.

US 2011/0112314 comprises a process for preparing olefins from oxygen-containing feeds.

Mixing gaseous or liquid hydrocarbons with high-temperature offgases is likewise known. Such high-temperature offgases contain carbon dioxide and/or water vapor and originate, in particular, from the converter. Mixing in hydrocarbons increases the proportion of carbon monoxide and of hydrogen in a reforming reaction with consumption of heat, thus also increasing the joule value of the offgas.

It is an object of the present invention to develop alternative processing of an offgas of the type mentioned at the outset. A further object of the present invention is to obtain products of value from such offgases. Greenhouse gases such as carbon dioxide, carbon monoxide and methane present in such offgases should not go into the atmosphere but instead be converted as completely as possible into products of value.

This object is achieved by the combination of features in independent claim 1. Further advantageous embodiments of the invention are indicated in the dependent claims.

In the process of the invention for processing offgas from a steelworks and/or a smelting works, wherein the offgas contains carbon dioxide and/or carbon monoxide, the offgas is fed, at least partly together with a hydrogen-containing gas, to a process for forming dimethyl ether, as a result of which a DME-containing product gas is formed.

According to the invention, the DME-containing product gas is fed to a process for converting dimethyl ether into olefins, resulting in formation of an olefin-containing product gas, and olefins, in particular ethylene and/or propylene, are separated off from the olefin-containing product gas by means of a separation process.

Furthermore, according to the invention, a ratio of hydrogen to carbon monoxide weighted by the carbon dioxide concentration

$$\frac{c [ H 2 ] - c [ CO 2 ]}{c [ CO ] + c [ CO 2 ]}$$

of from 0.9 to 1.1 is set at the inlet of the process for forming dimethyl ether, and dimethyl ether is formed. Carbon dioxide is advantageously also formed from carbon monoxide.

Here, the hydrogen content is regulated in such a way that the reaction proceeds selectively for dimethyl ether, depending on the further specific process (catalyst, etc.) for the formation of olefins, in particular ethylene.

The olefin-containing product gas according to the invention is either fed into the fractionation part of an existing olefin plant, where ethylene and/or propylene are likewise separated off as products of value, or the main products of value ethylene and/or propylene are isolated from the olefin-containing product gas in a separate separation plant.

The fundamental concept of the invention is that the offgases are not regarded exclusively as feeds to processes for generating electric energy, for example by means of a gas turbine. According to the invention, carbon dioxide, carbon monoxide and/or methane in the offgas are not only converted into dimethyl ether in order to increase the joule value. The DME-containing product gas obtained is fed as starting material into a process for producing olefins, so as to give an olefin-containing product stream. The olefins, in particular ethylene and propylene, are separated off from this product stream by means of known separation processes and obtained as product(s) of value. At least part of the offgas is mixed into a hydrogen-containing gas before the process for producing dimethyl ether in order to ensure that the hydrogen content in the feed to the process for producing dimethyl ether is sufficiently high to convert virtually all carbon oxides into dimethyl ether and obtain a high proportion of the two materials in the DME-containing product gas.

In principle, a type of synthesis gas composed of carbon monoxide and hydrogen is produced by the process of the invention by mixing of at least part of the offgas with a hydrogen-containing gas. According to the invention, at least part of the offgas is mixed with the hydrogen-containing gas. The amount of offgas to be mixed with the hydrogen-containing gas depends on the economic circumstances at the respective site. In the case of a sufficiently large amount of inexpensive and available hydrogen, the offgas obtained can all be mixed with the hydrogen-containing gas, with hydrogen-containing gas being able to be mixed in in such an amount that even carbon dioxide present in the offgas is converted virtually completely into carbon monoxide. If only a small amount of inexpensive hydrogen is available, this is utilized virtually completely and only a corresponding part of the offgas is mixed with the hydrogen-containing gas.

Process for converting, for example, methanol into olefins (e.g. production of ethylene by catalytic dehydrogenation of methanol over aluminum and zeolite catalysts) are known in the prior art and are described, for example, in “Ethylene”, H. Zimmermann and R. Walzl in Ullmann's Encyclopedia of Industrial Chemistry, Wiley 2011. The same applies to the separation of olefins, in particular ethylene and propylene, from such olefin-containing streams (see same reference and the references present therein). However, the present invention is not limited to the processes described there and the separation processes described there.

Basically, the invention combines processes for steel production and processes for obtaining olefins, in particular ethylene. According to the invention, all suitable constituents of the offgases from steelworks and/or smelting works are converted by means of hydrogen into dimethyl ether and, in a further step, olefins, in particular ethylene, are formed from dimethyl ether and are then separated off as product of value from the offgas. Thus, according to the present invention, products of value are obtained from the offgases and the offgases are not only optimized in respect of the joule value and used to generate electric energy as in the prior art.

The offgas is preferably discharged from a blast furnace and/or a converter and/or from a direct reduction process for iron ore. As mentioned at the outset, the offgas known as top gas from blast furnaces contains, like the offgas from converters, mainly carbon dioxide and carbon monoxide, so that both offgases are suitable for the present invention. The offgases can be treated separately or preferably together by means of the process of the invention.

Offgases from a direct reduction process for iron ore are particularly suitable for the process of the invention. Offgases from the direct reduction process for iron ore contain carbon monoxide and hydrogen in a ratio which is very particularly suitable for preparing dimethyl ether.

It is particularly advantageous to feed coking plant offgas as hydrogen-containing gas together with the offgas to the process for forming dimethyl ether. This embodiment of the invention is particularly suitable for integrated smelting works or steelworks. As mentioned above, coke is produced from coal in a coking plant. The coking plant gas formed in the coke oven contains hydrogen (about 55% by volume), methane (about 20% by volume), nitrogen, carbon monoxide, carbon dioxide, sulfur-containing compounds and higher hydrocarbons. The coking plant gas is therefore particularly suitable as hydrogen-containing gas, since, firstly, it is present in the integrated smelting works or steelworks and, secondly, it contains carbon dioxide, carbon monoxide and methane which can be converted catalytically into dimethyl ether.

In an embodiment of the invention, the offgas containing carbon monoxide and/or carbon dioxide and/or the hydrogen-containing gas are/is purified before the two are fed as feed into the process for forming dimethyl ether. Here, for example, all constituents except for carbon monoxide and/or carbon dioxide can be removed from the offgas. After purification, the hydrogen-containing gas advantageously consists of only hydrogen and optionally carbon monoxide and/or carbon dioxide.

Advantageously, the olefin-containing product gas is, after separating off the olefins, recirculated as alkane-containing tailgas for bottom firing to the coke oven and/or blast furnace. In the offgases from the furnaces, a small proportion of hydrocarbons (mainly alkanes) is firstly present, and secondly alkanes are also formed in secondary reactions in the formation of olefins. After the olefins, in particular ethylene and/or propylene, have been separated off from the olefin-containing product gas, the alkane-containing tailgas now consists mainly of alkanes and other combustible constituents. It is therefore very well suited for bottom firing of the furnaces (coke oven and/or blast furnace).

In one embodiment, methane is separated off from the alkane-containing tailgas and fed as feed into a gas turbine for generating electric energy. This embodiment of the invention combines the invention with the prior art in which the offgas is used mainly for generating electric energy. Among the constituents of the offgas, methane is best suited for use in a gas turbine for generating electric energy and is, in this embodiment of the invention, separated off from the alkane-containing tailgas and fed as feed into a gas turbine or fed into an existing natural gas grid.

In an alternative embodiment of the invention, a fraction containing hydrocarbons having not more than one carbon atom is separated off from the DME-containing product gas after the process for forming dimethyl ether. This fraction consists essentially of methane in this embodiment of the invention.

Hydrogen is advantageously separated off from the olefin-containing product gas by means of a cryogenic separation process. If the olefin-containing product gas still contains hydrogen which has not been reacted in the preceding process steps, this is automatically separated off by means of the cryogenic separation sequence (for example when the olefin-containing product gas is fed into an existing olefin plant or else in a separate separation sequence) and can be used as product in other places in the plant or be discharged.

In a further embodiment of the invention, the alkane-containing tailgas is fed to a process for the partial oxidation of alkanes to alkenes and alkynes in the presence of oxygen, resulting in formation of an oxidation product gas, and the oxidation product gas is recirculated to the separation process for separating off the olefins. The hydrogen and the oxidation product gas are advantageously fed to a process for the catalytic hydrogenation of alkynes, as a result of which a hydrogenation product gas is formed, and the hydrogenation product gas is recirculated to the separation process for separating off the olefins.

The recycle streams described likewise contain olefins, in particular ethylene and/or propylene, which further increase the ethylene and/or propylene yield and thus improve the economics.

In another embodiment of the invention, the alkane-containing tailgas is fed to a thermal process in the absence of oxygen, as a result of which a pyrolysis product gas and carbon are formed, and the pyrolysis product gas is fed to a pressure swing absorption process where it is separated into hydrogen and an acetylene-containing tailgas. The acetylene-containing tailgas consists very predominantly of acetylene which is discharged as product of value or can be used as fuel in the plant. Apart from the use of a pressure swing absorption process, alternative processes with which a person skilled in the art will be familiar, e.g. membrane separation processes or, particularly in the case of relatively high acetylene contents, chemical scrubbing comprising at least one scrubbing and regeneration step, are also conceivable.

The hydrogen is advantageously utilized as hydrogen product in other parts of the steelworks, the coking plant and/or the smelting works and/or outside these works.

In a further embodiment of the invention, the coking plant offgas is fed into a process for reforming methane to form carbon monoxide upstream of the process for forming dimethyl ether, forming a reformer product gas. In this embodiment of the invention, the carbon monoxide content at the inlet of the process for forming dimethyl ether is increased and formation of the product in this process is thus promoted. Thus, more olefins, in particular ethylene and/or propylene, can be formed in the subsequent process step. In addition, the methane content of the olefin-containing product gas becomes lower and the isolation of the olefins, in particular ethylene and/or propylene, is thus simplified. In an alternative embodiment, the reformer can be combined with a water gas shift reactor.

The alkane-containing tailgas can likewise be recirculated together with the offgas to the process for reforming methane in order to increase the proportion of carbon monoxide upstream of the process for forming dimethyl ether.

In an alternative embodiment of the invention, the offgas is at least partly fed into a process for removing carbon dioxide, nitrogen and/or methane upstream of the process for forming dimethyl ether and before mixing-in of hydrogen. This embodiment of the invention is particularly useful for sites having a small amount of inexpensively available hydrogen. This embodiment of the invention is especially advantageous at sites having a small amount of inexpensive hydrogen. In this embodiment of the invention, potential hydrogen consumers are removed from the subsequent process steps. Thus, for example, carbon dioxide reacts with hydrogen to form carbon monoxide and water under suitable conditions or in the presence of catalysts. As a result of the removal of hydrogen consumers in this embodiment of the invention, a relatively pure synthesis gas composed of carbon monoxide and hydrogen is introduced into the process for forming dimethyl ether and the subsequent process steps.

The present invention makes it possible to provide, in particular, an alternative process of the type mentioned at the outset. Pollution of the environment by carbon monoxide or carbon dioxide from smelting works and/or steelworks is minimized, and products of value such as ethylene and/or propylene are at the same time obtained from the offgases. The economics of operation of smelting works and/or steelworks are therefore improved by means of the present invention.

The invention is illustrated below with the aid of the examples shown in the figures. The process schemes shown in the figures in each case describe the process of the invention in an integrated steelworks comprising coke oven, blast furnace and converter. Here, identical parts/process steps have been denoted by identical reference numerals.

The figures show

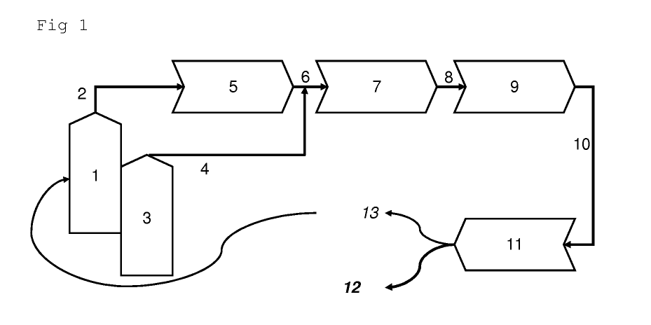

FIG. 1 an example with methane reforming,

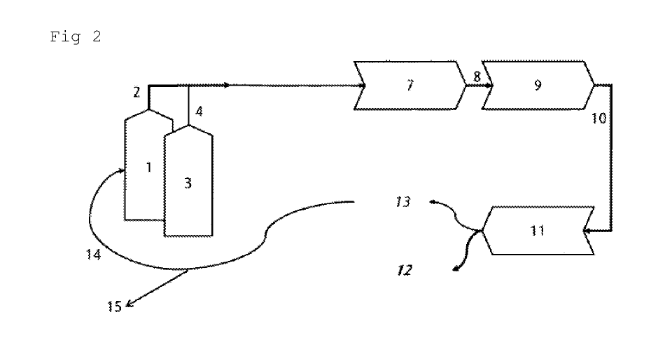

FIG. 2 an example without methane reforming,

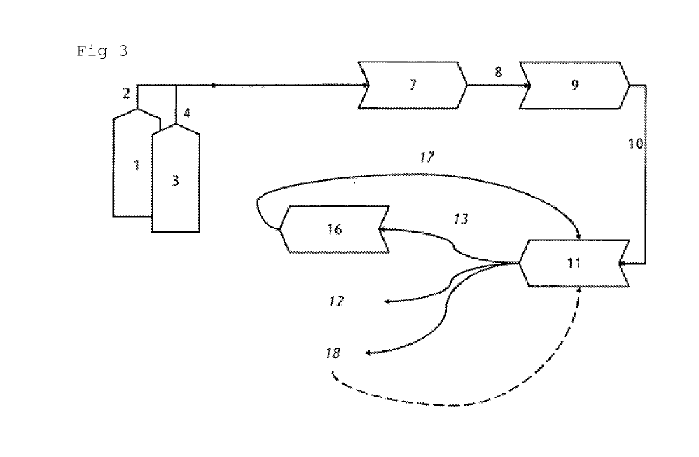

FIG. 3 an example with partial oxidation of the alkane-containing tailgas and

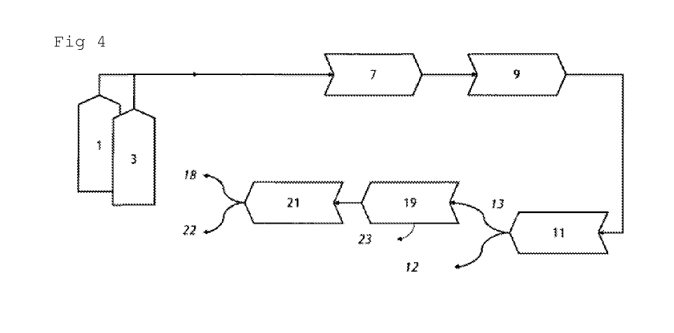

FIG. 4 an example with oxygen-free pyrolysis of the alkane-containing tailgas.

FIG. 1 shows a rough process scheme of an example with methane reforming in an integrated steelworks comprising coke oven, blast furnace and converter.

In the coke oven 1, carbon is pyrolyzed to coke in the absence of oxygen. The coking plant gas 2 formed here contains carbon monoxide, carbon dioxide and methane in addition to the main constituent hydrogen. The coking plant gas 2 is introduced into a reforming stage 5 for converting methane into carbon monoxide, as a result of which a reformer product gas 6 is formed. The reformer product gas 6 is virtually free of methane and has a significantly higher proportion of carbon monoxide than the coking plant gas 2.

In the converter 3, the carbon is removed from the pig iron and steel is produced. The offgas 4 formed here is mixed with reformer product gas 6, a hydrogen-containing gas, and is introduced as feed into a process 7 for forming dimethyl ether, as a result of which a DME-containing product gas 8 is formed. In the process 7, mainly carbon monoxide and carbon dioxide are reacted with hydrogen to form dimethyl ether over a catalyst having one or more active sites.

The DME-containing product gas 8 is introduced as feed into a process 9 for converting dimethyl ether into olefins, in particular ethylene and/or propylene, as a result of which an olefin-containing product gas 10 is formed. In this process 8, the environmentally harmful constituents carbon monoxide and carbon dioxide in the offgas 4 were thus converted into products of value 12 which are separated off from the olefin-containing product gas 10 by means of separation process 11. Especially ethylene and/or propylene are obtained as products of value 12. The alkane-containing tailgas 13 remaining after the products of value 12 have been separated off consists mainly of alkanes and other combustible constituents and is recirculated 14 for bottom firing to the coke oven 1 or the blast furnace (not shown).

The example shown in FIG. 2 corresponds to the example in FIG. 1, but in this case a process for methane reforming was omitted. The offgas 4 from the converter 3 is mixed directly with the hydrogen-containing coking plant gas 2 and introduced as feed into a process 5 for forming dimethyl ether. Methane is inert in respect of the subsequent processes and is thus, in this embodiment of the invention, recirculated together with the alkane-containing tailgas 13 for bottom firing 14 to the coke oven 1 and the blast furnace 15.

The example shown in FIG. 3 is similar to the example in FIG. 2, but in this example of the invention the alkane-containing tailgas 13 is fed to a thermal process 16 for converting alkanes into alkenes and alkynes in the presence of oxygen, forming an oxidation product gas 17 which is recirculated to the separation process 11 for separating off the olefins 12. In the thermal pyrolysis process 16, alkenes and alkynes are formed in the optional presence of a catalyst. In this embodiment, hydrogen 18 is also separated off from the olefin-containing product gas 10 in cryogenic separation process 11. This can be utilized anywhere in the plant or be recirculated to the separation process 11 (broken line) in order to hydrogenate the alkynes in the oxidation product gas 17.

The example shown in FIG. 4 is similar to the example in FIG. 3, but according to this example of the invention the alkane-containing tailgas 13 is fed to a thermal process 19 for converting alkanes into alkenes and alkynes in the absence of oxygen, forming, in the absence of a catalyst, mainly acetylene and hydrogen.

This pyrolysis product gas 20 is fed to a process for pressure swing absorption 21 and separated into the main components hydrogen 18 and acetylene 22.

The hydrogen 18 can be used in any parts of the plant; in particular, use as reducing agent in the catalytic removal of nitrogen oxides from flue gases is possible.

1. A process for processing offgas (4) from a steelworks and/or a smelting works, wherein the offgas (4) contains carbon dioxide and/or carbon monoxide and is fed, at least partly together with a hydrogen-containing gas (2, 6) to a process (7) for forming dimethyl ether, as a result of which a DME-containing product gas (8) is formed, a ratio of hydrogen to carbon monoxide weighted by the carbon dioxide concentration
$$\frac{c [ H 2 ] - c [ CO 2 ]}{c [ CO ] + c [ CO 2 ]}$$
of from 0.9 to 1.1 is set at the inlet of the process (7) for forming dimethyl ether, the DME-containing product gas (8) is fed to a process (9) for converting dimethyl ether into olefins, as a result of which an olefin-containing product gas (10) is formed, and olefins (12), in particular ethylene and/or propylene, are separated off from the olefin-containing product gas by means of a separation process (11).

2. The process as claimed in claim 1, characterized in that the offgas (4) is discharged from a blast furnace and/or a converter and/or from a direct reduction process for iron ore.

3. The process as claimed in claim 1, characterized in that coking plant offgas (2), in particular offgas (2) from a coke oven, is fed as hydrogen-containing gas together with at least a part of the offgas (4) into the process (7) for forming dimethyl ether.

4. The process as claimed in claim 1, characterized in that a ratio of hydrogen to carbon monoxide weighted by the carbon dioxide concentration
$$\frac{c [ H 2 ] - c [ CO 2 ]}{c [ CO ] + c [ CO 2 ]}$$
of 1 is set at the inlet of the process (7) for forming dimethyl ether.

5. The process as claimed in claim 1, characterized in that the olefin-containing product gas (10) is, after separating off the olefins (12), recirculated (14) as alkane-containing tailgas (13) for bottom firing to the coke oven and/or blast furnace.

6. The process as claimed in claim 1, characterized in that methane (15) is separated off from the alkane-containing tailgas (13) and fed as feed into a gas turbine for generating electric energy.

7. The process as claimed in claim 1, characterized in that hydrogen (18) is separated off from the olefin-containing product gas by means of a cryogenic separation process (11).

8. The process as claimed in claim 1, characterized in that the alkane-containing tailgas (13) is fed to a process for the partial oxidation (16) of alkanes to alkenes and alkynes in the presence of oxygen, resulting in formation of an oxidation product gas (17), and the oxidation product gas (17) is recirculated to the separation process (11) for separating off the olefins (12).

9. The process as claimed in claim 8, characterized in that the hydrogen (18) and the oxidation product gas (17) are fed to a process for the catalytic hydrogenation of alkynes, as a result of which a hydrogenation product gas is formed, and the hydrogenation product gas is recirculated to the separation process (11) for separating off the olefins.

10. The process as claimed in claim 1, characterized in that alkane-containing tailgas (13) is fed to a thermal process (19) in the absence of oxygen, as a result of which a pyrolysis product gas and carbon (23) are formed, and the pyrolysis product gas is fed to a pressure swing absorption process (21) where it is separated into hydrogen (18) and an acetylene-containing tailgas (22).

11. The process as claimed in claim 1, characterized in that the hydrogen (18) is utilized as hydrogen product in other parts of the steelworks, the coking plant and/or the smelting works and/or outside these works.

12. The process as claimed in claim 3, characterized in that coking plant offgas (2) is fed into a process (5) for reforming methane to form carbon monoxide upstream of the process for forming dimethyl ether, forming a reformer product gas (6).

13. The process as claimed in claim 1, characterized in that the offgas (4) is fed into a process for removing carbon dioxide, nitrogen and/or methane upstream of the process (7) for forming dimethyl ether.

14. (canceled)
